(19) United States
(12) Patent Application Publication
(10) Pub. No.: US 2026/0130722 A1
(43) Pub. Date: May 14, 2026

(54) USER ASSISTANCE SYSTEM FOR NAVIGATION OF A MEDICAL DEVICE

(71) Applicant: Siemens Healthineers AG, Forchheim (DE)

(72) Inventors: Markus Kowarschik, Erlangen (DE); Rebecca Fahrig, Mohrendorf (DE)

(21) Appl. No.: 19/375,720

(22) Filed: Oct. 31, 2025

(30) Foreign Application Priority Data
Nov. 14, 2024 (DE) 10 2024 210 932.0

(51) Int. Cl. A61B 34/20; A61B 90/00; G06F 3/01; G06T 7/00; G16H 40/63

(52) U.S. Cl. CPC A61B 34/20; A61B 90/37; G06F 3/013; G06T 7/0012; G16H 40/63; A61B 2034/2065; G06T 2207/30004

(57) ABSTRACT

A user assistance system for image support during navigation of a medical device includes an imaging system configured, for image support during navigation of the medical device, to generate image data that at least partially maps the medical device and to display a graphical representation of the image data. Furthermore, the user assistance system includes a monitoring system configured to monitor whether the medical device is moving and initiate a first risk-reducing measure when the image support by the imaging system is deactivated and, according to the monitoring by the monitoring system, the medical device is moving at the same time.

The present patent document claims the benefit of German Patent Application No. 10 2024 210 932.0, filed Nov. 14, 2024, which is hereby incorporated by reference in its entirety.

### TECHNICAL FIELD

The present disclosure relates to a user assistance system and a method for image support during navigation of a medical device. The user assistance system includes an imaging system configured, for image support during navigation of the medical device image, to generate and display image data that at least partially maps the medical device.

### BACKGROUND

Within vascular surgery, the need for minimally invasive procedures is constantly increasing due to a variety of clinical pictures. Herein, endovascular surgery plays a central role, as it is an essential component of modern vascular surgery in the treatment of peripheral or central arterial occlusive diseases.

Endovascular surgery relies on the use of medical devices such as guidewires and catheters that are inserted into a patient's vascular system to diagnose or treat pathologies that may be reached via such access routes. For example, the devices are mapped in the patient's body using X-rays, in particular using angiographic C-arm systems, in order to assist a user and/or a robotic system during navigation of the medical devices.

Clinical applications include, inter alia, cardiology (for example, treatment of heart attacks by stenting), radiology/oncology (for example, targeted administration of a chemotherapeutic agent to a tumor in the liver of a patient), and neuroradiology (for example, mechanical thrombectomy for the treatment of ischemic stroke).

Current development activities are focused on robotic systems for maneuvering endovascular devices in the vascular system of the patient. Development goals include improving patient safety, standardizing treatment quality with the associated reduction in complication rates and increasing workflow efficiency.

One risk of endovascular surgery is injury due to suboptimal placement or movement of medical devices in the patient's vascular system. This risk may be significantly reduced by image support, thereby more reliably providing a successful outcome of the procedure. Therefore, image support may make a significant contribution to patient safety because the placement of the medical device may be visualized and thus controlled. Without this control, uncontrolled movements of the medical device may occur, potentially leading to injury.

### SUMMARY AND DESCRIPTION

The scope of the present disclosure is defined solely by the appended claims and is not affected to any degree by the statements within this summary. The present embodiments may obviate one or more of the drawbacks or limitations in the related art.

It is an object of the present disclosure to further reduce the risk of uncontrolled and/or suboptimal movement of a medical device.

The disclosure is based on the idea of initiating a risk-reducing measure as soon as a movement of a medical device is detected without active imaging being activated at the same time.

A first aspect of the disclosure presents a user assistance system for image support during navigation of a medical device. The user assistance system includes an imaging system, (e.g., a medical imaging system), which is configured, for image support during navigation of the medical device, to generate image data that at least partially maps the medical device, (e.g., in an object), and to display a graphical representation of the image data, (e.g., on a display unit). In addition, the user assistance system includes a monitoring system that is configured to monitor whether the medical device is moving. The monitoring system is configured to initiate a first risk-reducing measure if the image support by the imaging system is deactivated and, according to the monitoring by the monitoring system, the medical device is moving at the same time.

In other words, the monitoring system may monitor an image support state, e.g., determine whether the image support by the imaging system is activated or deactivated, and, on the other hand, monitor the movement of the medical device. As soon as the image support is deactivated, (e.g., no image data is output), and at the same time the movement of the medical device is detected, the first risk-reducing measure may be initiated. Herein, it may be the case that initially a first condition, namely deactivated image support, occurs first, or a second condition, namely detection of the movement of the medical device, occurs first. Herein, in some embodiments, the first measure may differ depending on the sequence, but, in other embodiments, they may be independent of the sequence.

A risk-reducing measure may refer to a measure that reduces the risk of injury due to uncontrolled or unintentional movement of the medical device. The risk-reducing measure may include a warning to a user, a deactivation of movement capability of the medical device, or an activation or reactivation of image support or another measure that may establish or restore normal operation for a procedure.

Normal operation may refer to an operation in which the imaging system generates the image data, (e.g., continuously, repeatedly, or periodically), and provides or displays the graphical representation of the image data on a display so that the user may recognize a map of the medical device against a background of an environment. In this case, the movement of the medical device may take place with little risk, or in particular no risk.

The monitoring system is configured to monitor the movement of the medical device. This monitoring may take place continuously, at regular temporal intervals, periodically, or repeatedly. In particular, the monitoring may only detect movement as movement if a minimum distance is covered by the medical device within a unit of time. Movements that fall below this limit may be ignored. This may prevent the first measure from being triggered repeatedly if the risk of injury may be eliminated.

Herein, navigation may be understood to be targeted movement of the medical device along a route. Herein, a destination of the movement may be at least one predetermined region that is to be reached with the medical device. Herein, for example, at least a part of the route may be visible to the user with the aid of the image support. In particular, part of the route may be unknown to the user and/or new destinations may arise along the route.

Navigation may be triggered or performed manually by the user by the user moving the medical device directly. Alternatively, a robotic system may control the navigation of the medical device and trigger the movement automatically, for example, by at least one actuator. Alternatively, or additionally to automation, the user may actuate the navigation of the medical device from a remote workstation, e.g., a workstation located in a different room from the medical device. Such actuation may also be referred to as remote operation.

Herein, there may be various reasons for deactivating image support. For example, there may be an error in the imaging system. Herein, the error may be an error in the generation of the image data or an error in the display of the graphical representation of the image data or other errors that cause the imaging system to malfunction. Likewise, there may also be an error in a component of the imaging system. In other words, image support by the imaging system does not necessarily have to be completely deactivated in order to initiate the first measure. It is also possible that the detecting or processing of data, in particular raw data, necessary for the image data may be deactivated or faulty. Likewise, the display unit may be deactivated or faulty. Likewise, a faulty or deactivated communication path to the robotic system may lead to deactivation of the image support.

In particular, the deactivation of the image support may also have been triggered by an intentional action, for example, by an intentional action on the part of the user.

Alternatively, this may also entail a partial or complete shutdown of the imaging system. Herein, the partial or complete shutdown may be triggered by the user or, for example, by a failure of a power supply of the imaging system or another error. Herein, a partial shutdown may also affect components of the imaging system that are configured to display the graphical representation of the image data, such as a display unit, e.g., a monitor. Accordingly, a described state of the imaging system is monitored by the monitoring system and, together with the movement of the medical device, may trigger the first measure.

The first measure may entail issuing a warning message, wherein the warning message may be issued specifically for the user of the user assistance system.

For example, in some embodiments, the user assistance system may include at least one actuator and thus exert an active influence on the movement of the medical device. Such a user assistance system may be a robotic system, e.g., a medical or endovascular robotic system. The first measure may also include deactivating the at least one actuator and thus terminating the movement of the medical device. In particular, the first measure may also correspond to deactivation of the at least one actuator.

A risk from the movement of the medical device may arise due to the proximity of the medical device to the user or to a patient. In particular, the movement may cause undesirable effects, incorrect or suboptimal placement of the medical device, or even injury to the user or patient, e.g., if the movement occurs without the image support providing feedback to the user, who may take corrective action if necessary.

The medical device may be an endovascular device or the medical device may include one or more endovascular devices. Herein, an endovascular device may be located at least partially in the body of a human or animal, in particular in a vascular structure or hollow organ. For example, undesirable movement of the endovascular device may cause perforated vessel walls or a ruptured aneurysm. Likewise, the movement may lead to other injuries, which, for example, may likewise be caused by part of the medical device that is located within the body of the human or animal. In some embodiments, the user assistance system may include the medical device.

The object may be the patient or a body part of the patient. The patient may be placed on a patient bench of the imaging system. The medical device may be located in the object or partially in the object.

The image data may be one or more items of X-ray projection image data, for example, a plurality of items of X-ray projection image data from different perspectives. Likewise, the graphical representation of the image data may be a three-dimensional reconstruction or a partial three-dimensional reconstruction, e.g., from cone beam computed tomography, digital volume tomography, or tomosynthesis. Alternatively, the image data may also originate from real-time magnetic resonance tomography.

One advantage of the disclosure presented here is the minimization of risks with regard to consequences that may be triggered by uncontrolled movement of the medical device. The user may be reliant on image support when using the medical device in regions that cannot be seen with the naked eye. If a situation arises in which this image support is not available or no longer available, the first measure is triggered, e.g., the user is warned as soon as the medical device moves. Likewise, an escalation may be defined by preventing the medical device from moving. This may make navigation more fail-safe and increase the success rate of navigation.

The monitoring system may include a data processing system. In the present disclosure, the terms “data processing system” and “at least one data processing device” may be used interchangeably. A data processing device may be understood to mean a data processing device including a data processing circuit. Therefore, the data processing device may therefore process data for performing computing operations. These may also include operations for performing indexed access to a data structure, for example, a look-up table (LUT), as well as a data processing process implemented in hardware.

The data processing device may include one or more computers, one or more microcontrollers, and/or one or more integrated circuits, for example, one or more application-specific integrated circuits (ASICs), one or more field-programmable gate arrays (FPGAs), and/or one or more systems on a chip (SoCs). The data processing device may also include one or more processors, for example, one or more microprocessors, one or more central processing units (CPUs), one or more graphics processing units (GPUs), and/or one or more signal processors, in particular one or more digital signal processors (DSPs). The data processing device may also include a physical or virtual network of computers or other examples of the aforementioned units.

In various exemplary embodiments, the data processing device includes one or more hardware and/or software interfaces and/or one or more memory units.

A memory unit may be a volatile data memory, for example, a dynamic random access memory (DRAM) or static random access memory (SRAM), or a non-volatile data memory, for example, read-only memory (ROM), programmable read-only memory (PROM), erasable programmable read-only memory (EPROM), an electrically erasable programmable read-only memory (EEPROM), a flash memory or flash EEPROM, a ferroelectric random access memory (FRAM), a magnetoresistive random access memory (MRAM), or a phase-change random access memory (PCRAM).

According to at least one embodiment, the imaging system is embodied as a medical imaging system for mapping the medical device in an organ and/or tissue.

Herein, the imaging system may map at least one part of the medical device, in particular an end of the medical device. The medical device may be a guidewire and/or a catheter, for example, for use in endovascular surgery, a vascular implant, a stent, a flow diverter, and so on, or a combination of such devices, which may also be referred to as a device stack.

At the time of mapping, the medical device may be located in the organ and/or tissue of a human or animal body, in particular in a hollow organ.

Herein, the medical imaging system may be an X-ray system, a fluoroscopy system, an angiography system, a magnetic-resonance tomography system, or an ultrasound system, or the medical imaging system may include one or more such imaging modalities. Herein, in particular, part of the human or animal body in which the medical device is located at the respective time may be mapped locally.

One advantage of such embodiments is a true-to-detail representation of the medical device by the medical imaging method. In particular, it is possible to represent regions of the human or animal body that would be difficult or impossible to recognize without such a medical imaging method. Thus, the medical imaging method also enables image support in this case, thereby facilitating navigation of the medical device in the organ, in particular in the hollow organ.

According to at least one further embodiment, the monitoring system is configured to check an image content of the image data and/or the graphical representation of the image data for a predetermined criterion. In addition, the monitoring system is configured to initiate a second risk-reducing measure if the image content does not meet the predetermined criterion and, according to the monitoring by the monitoring system, the medical device is moving at the same time.

In other words, the data processing system may check the image content of the image data and/or the graphical representation of the image data for the predetermined criterion. For this purpose, the data processing system may receive the image data from the imaging system and be configured to analyze the image content or to process the image.

Herein, the predetermined criterion may be a predetermined quality criterion for the image content of the image data and/or the graphical representation of the image data. For example, the quality criterion may depend on a signal-to-noise ratio, contrast ratio, image resolution, image sharpness, freedom from artifacts and/or freedom from distortion. Herein, a specification may be used for uniquely identifying the medical device in its immediate environment, e.g., against a vascular background. The quality criteria may be particularly relevant in the case of small medical devices or small structures, such as guidewires.

Alternatively, or additionally, the predetermined criterion may also relate to checking that the medical device is present. In other words, a specification may be that the medical device or a predefined part of the medical device is represented by the image content, e.g., that a relevant part of the human or animal body is mapped. Herein, the medical device may be selected from a list of medical devices, so that the criterion may be defined based on the shape of the respective medical device. In this way, the second measure may be initiated if the medical device may not be identified or may not be clearly identified in the image content and movement is present.

The second measure may likewise include the options already described for the first measure, in particular the issuing of a warning message and/or the deactivation of the at least one actuator. The second measure may be identical to the first measure or differ therefrom.

Taking account of the predetermined criterion is particularly advantageous when image support is activated, but, due to reduced image quality or other reasons, it is objectively impossible to identify the medical device or only possible to identify it to a limited extent or ambiguously. This may prevent missing information in the image content from leading to uncontrolled movement.

According to at least one further embodiment, the user assistance system includes a gaze detection system that is configured to detect the gaze direction of a user. In addition, the monitoring system is configured to initiate a third risk-reducing measure and/or a fourth risk-reducing measure depending on a result of the detection of the gaze direction.

In other words, the gaze detection system may include a camera that is directed at a position where the user is located. This may be a workstation configured to work with the user assistance system. For this purpose, the camera may be configured to detect an image of the face of the user and in particular to identify the eyes. The gaze direction may be detected depending on the alignment of the pupils. For example, the gaze detection system may be configured for further image processing. The gaze detection system may also be referred to an eye tracker.

The further image processing of the gaze detection system may be part of the data processing system or coupled thereto. In particular, the image processing may be configured to analyze the image of the face and recognize the eyes in the image and determine the gaze direction of the user.

In particular, prior calibration at a predetermined camera position enables the gaze direction of the user to be detected unambiguously and, for example, assigned to other objects that are likewise located at a predetermined position. For example, a display unit on which the graphical representation of the image data is displayed for image support, (e.g., a monitor), may be located at the predetermined position and the gaze detection system may detect whether the display unit is in the gaze direction of the user.

One advantage of such embodiments is the ability to check attention of the user. The gaze direction may enable conclusions to be drawn about user behavior and lead to a targeted warning message and/or deactivation of the movement of the medical device, which may be helpful to the user during navigation.

According to at least one further embodiment, the monitoring system is configured to initiate the third risk-reducing measure if the gaze detection system does not detect the gaze direction of the user and, according to the monitoring by the monitoring system, the medical device is moving at the same time, or the monitoring system is configured to initiate the third risk-reducing measure if the gaze direction of the user cannot be detected by the gaze detection system and, according to the monitoring by the monitoring system, the medical device is moving at the same time.

In other words, the third measure may be initiated if the gaze detection system does not detect the face of the user or the eyes of the user or the pupils of the user and the medical device is moving. This may be the case if the user is not at the workstation or the back of the user is turned to the gaze detection system, for example, the camera, or if the face or the pupils of the user cannot be recognized in the image section.

The third measure may likewise include the options already described for the first measure, in particular the issuance of a warning message and/or the deactivation of the at least one actuator. The third measure may be identical to the first measure or differ therefrom. The third measure may be identical to the second measure, if provided, or differ therefrom.

One advantage of such embodiments is that the embodiments provide correct use of the user assistance system. For example, the position of the display unit may be used to determine where the user may operate the assistance system. In this position, the gaze detection system may detect the gaze direction. An undetected gaze direction may then indicate improper use and may lead to uncontrolled movement of the medical device. This may be avoided with the embodiment.

According to a further embodiment, the user assistance system includes a display unit that is configured to display the graphical representation of the image data.

According to a further embodiment, the monitoring system is configured to initiate a fourth risk-reducing measure if the gaze detection system determines that the gaze direction of the user is not directed at the display unit and, according to the monitoring by the monitoring system, the medical device is moving at the same time.

The gaze detection system may include the camera that is configured to detect the gaze direction of the user, for example, by evaluating the image of the face or the pupils of the user. The camera may be arranged above the monitor in order to detect the image at the workstation.

The fourth measure may likewise include the options already described for the first measure, in particular the issuance of a warning message and/or the deactivation of the at least one actuator. The fourth measure may be identical to the first measure or may differ therefrom. The fourth measure may be identical to the second measure, if provided, or may differ therefrom. The fourth measure may be identical to the third measure, if provided, or may differ therefrom.

One advantage of such embodiments is that the embodiments specifically provide correct use of the user assistance system. If the gaze direction of the user is not directed at the display unit, the user may not be able to follow the movement of the medical device. The embodiment may increase system safety.

According to a further embodiment, the user assistance system includes at least one actuator which is configured to move the medical device.

In other words, the user assistance system may trigger or control an automatic movement of the medical device with the aid of the at least one actuator. The automatic movement by the at least one actuator may be referred to as robotics, in particular endovascular robotics. Herein, for example, the user may actuate the at least one actuator with the aid of the user assistance system.

Likewise, the movement may be triggered manually, in particular by the user.

One advantage of such embodiments is that the movement of the medical device may be performed more precisely and reproducibly. Automatic movement by the robotics also enables a physical separation between the medical device, which is, for example, located in the human or animal body, and the user. If the movement is controlled by an imaging system based on X-rays, the physical separation would protect the user from the X-rays to a large extent.

According to a further embodiment, the user assistance system includes a control device that is configured to generate at least one control signal for actuating the at least one actuator, in particular to automatically move the medical device. In addition, the monitoring system is configured, depending on the control signal, to monitor whether the medical device is moving.

In particular, the control device may generate at least one control signal for each individual actuator of the at least one actuator. This allows each individual actuator to be actuated individually and monitored if necessary.

In some embodiments, the control device may include a user input device, e.g., a switch, control lever, or joystick. The control device may then generate the at least one control signal from the input signal of the user input device and thereby actuate the at least one actuator. Likewise possible are fully automatic sequences in which the control device generates the at least one control signal without an input signal being present. This may be the case with predetermined routes in which no individual adaptation is necessary.

Thus, the at least one control signal may be an indicator of the movement of the medical device. Hence, the monitoring system may determine the movement of the medical device based on the presence of the at least one control signal.

One advantage of such embodiments is a precise and immediate determination of the movement of the medical device. At a point in time when the at least one control signal is generated and a second condition relating to image support is present, one of the measures may already be initiated with little time delay, or no time delay.

According to a further embodiment, the first measure includes deactivating the at least one actuator.

In other words, the deactivation of the at least one actuator may enable the first measure to stop the automatic movement of the medical device, e.g., to bring the medical device to a standstill.

The monitoring system may be connected to the control device and be configured to deactivate the at least one control signal with the aid of the control device within the first measure. Herein, in particular, the first measure may result in the control device no longer issuing a control signal or the at least one control signal transferring the at least one actuator to a rest position.

Likewise, the second measure, the third measure and/or the fourth measure may include deactivating the at least one actuator. In particular, each of the measures may correspond to deactivation.

One advantage of such embodiments is the targeted stopping of the movement of the medical device. Initiating the first measure or initiating the second, third or fourth measure enables the risk of uncontrolled movement to be avoided by preventing automatic movement. In particular, the deactivation of the at least one actuator may be an escalation stage of the first measure, e.g., after an unsuccessful issuance of a warning.

According to a further embodiment, the user assistance system includes at least one sensor which is configured to issue a sensor signal depending on the movement of the medical device. In addition, the monitoring system is configured to monitor whether the medical device is moving depending on the sensor signal.

The at least one sensor may include at least one acceleration sensor. The at least one sensor may be connected to the medical device or be part of the medical device. In some embodiments, the user assistance system may include the medical device.

The at least one sensor may also include a camera and/or a motion detector, which are each configured to detect the movement of the medical device and to issue the sensor signal depending on the movement of the medical device. Herein, an image signal or a video stream from the camera may also be regarded as at least one sensor signal.

Thus, the at least one sensor signal may be a further indicator of the movement of the medical device. Hence, the monitoring system may determine the movement of the medical device based on the presence of the at least one sensor signal.

The monitoring system may also receive a combination of the at least one sensor signal and the at least one control signal and be configured to monitor both signals independently of one another.

One advantage of such embodiments is a precise and immediate determination of the movement of the medical device, in particular if the medical device may be moved manually, e.g., by the user.

According to a further embodiment, the first measure includes issuing at least one warning signal.

Likewise, if provided, the second measure, the third measure and/or the fourth measure may include issuing the at least one warning signal. In particular, each of the measures may correspond to the issuance.

One advantage of such embodiments is the timely transfer of information to the user and thus further risk reduction and avoidance of uncontrolled movement of the medical device.

According to a further embodiment, the at least one warning signal includes a visual signal and the user assistance system is configured to display the visual signal.

In other words, the user assistance system may be connected to the display unit and be configured to issue the visual signal on the display unit. Herein, the display unit may be the monitor and the visual signal may be a warning message in the foreground of the monitor. Herein, the display unit may be configured to depict the graphical representation of the image data of the medical device generated by the imaging system in the background.

Likewise, the visual signal may include or correspond to a flashing warning light. Herein, in particular, the warning light may be located in the field of view of the user.

One advantage of such embodiments is the rapid and unambiguous communication of necessary information to the user. In particular, the user may also be specifically alerted to key points in order to minimize or avert further risks.

According to a further embodiment, the display unit is configured to display the visual signal at a display position, in particular at a display position of the display unit, which lies in the detected gaze direction of the user.

In other words, the display unit may be configured to display the visual signal, for example, as a warning message on the monitor at the display position that the user is viewing at the time. In particular, the user assistance system may be configured to communicate the gaze direction of the gaze detection system to the display unit. For example, the location of the display unit may be specified so that the display position of the visual signal may match the gaze direction of the user. In particular, upstream calibration or adaptation to individual user behavior or, for example, the respective workstation may be necessary in order to find a correct display position.

One advantage of such embodiments is targeted and clearly visible communication of necessary information to the user via the visual signal at the point of the gaze direction of the user. This reduces the likelihood of the visual signal being accidentally overlooked.

According to a further embodiment, the at least one warning signal includes an acoustic signal and the user assistance system is configured to issue the acoustic signal.

Herein, the user assistance system may be configured to issue the acoustic signal in the immediate environment of the user. In particular, the at least one warning signal may include both an acoustic signal and a visual signal. Herein, the acoustic signal may include different tones which, depending on the intensity of the movement of the medical device or the frequency of occurrence of the at least one warning signal, swell, become louder, or become more penetrating due to a tone frequency. The acoustic signal may also include voice output.

In particular, the at least one warning signal may differ for the first, second, third, or fourth measure.

One advantage of such embodiments is the clear communication of necessary information to the user. Herein, in particular, the attention of the user may be obtained, thereby reducing the risk of uncontrolled movement.

In a further aspect, a method is presented for image support during navigation of a medical device by displaying a graphical representation of image data generated by an imaging system, wherein the graphical representation at least partially maps the medical device. Herein, a monitoring system monitors whether the medical device is moving. In addition, a first risk-reducing measure is initiated if the image support is deactivated and, according to the monitoring by the monitoring system, the medical device is moving at the same time.

According to at least one embodiment of the method, the imaging system is embodied as a medical imaging system and the medical device is located in an organ.

In particular, the navigation of the medical device is not part of the method. The movement of the medical device or the like is also not part of the method. The method does not include any surgical acts, because the method is intended solely for user assistance.

Further embodiments of the method emerge directly from the various embodiments of the user assistance system and vice versa. In particular, individual features and corresponding explanations and advantages relating to the various embodiments of the user assistance system may be transferred analogously to corresponding embodiments of the method. In particular, the user assistance system is embodied or programmed to perform a method. In particular, the user assistance system performs the method as described herein.

Further features and combinations of features of the disclosure emerge from the figures and their description and from the claims. In particular, further embodiments of the disclosure do not necessarily have to include all the features of one of the claims. Further embodiments of the disclosure may have features or combinations of features that are not mentioned in the claims.

The disclosure is explained in more detail in the following with reference to specific exemplary embodiments and associated schematic drawings. In the figures, identical or functionally identical elements may be provided with the same reference symbols. The description of the identical or functionally identical elements is not necessarily repeated for different figures.

### BRIEF DESCRIPTION OF THE DRAWINGS

FIG. 1 depicts a schematic representation of an exemplary embodiment of a user assistance system.

FIG. 2 depicts a schematic block diagram of a further exemplary embodiment of a user assistance system for image support during navigation of a medical device.

FIG. 3 depicts a schematic block diagram of a further exemplary embodiment of a user assistance system for image support during navigation of a medical device.

### DETAILED DESCRIPTION

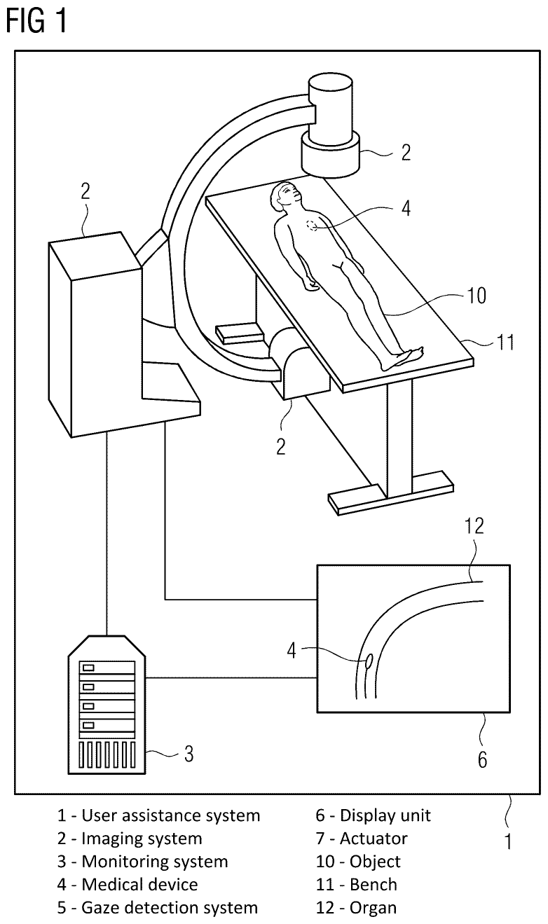

FIG. 1 is a schematic view of an exemplary embodiment of a user assistance system 1. The user assistance system 1 has an imaging system 2 that is configured to generate a graphical representation of image data of an object 10, wherein the graphical representation at least partially maps a medical device 4.

The imaging system 2 is shown by way of example as a C-Arm X-ray device. However, the following explanations may be transferred analogously to other imaging systems 2 that may generate images depicting the medical device 4, in particular in an object 10.

The object 10 may be a patient or a body part of the patient. The patient may be placed on a patient bench 11 of the imaging system 2. The medical device 4 may be located in the object 10 or partially in the object 10.

Likewise shown is a monitoring system 3 of the user assistance system 1 which is connected to the imaging system 2 so that the monitoring system 3 may receive information about an operating status of the imaging system 2. Furthermore, a display unit 6 is provided, which may depict the graphical representation of the image data of the imaging system 2. By way of example, the medical device 4 is depicted in the object 10, in particular in an organ 12 or a hollow organ of the patient. The display unit 6 may be part of the user assistance system 1, in particular of the monitoring system 3.

The monitoring system 3 is configured to monitor whether the medical device 4 is moving. In addition, the monitoring system 3 is configured to initiate a first risk-reducing measure 101 if the image support by the imaging system 2 is deactivated and, according to the monitoring by the monitoring system, 3 the medical device 4 is moving at the same time.

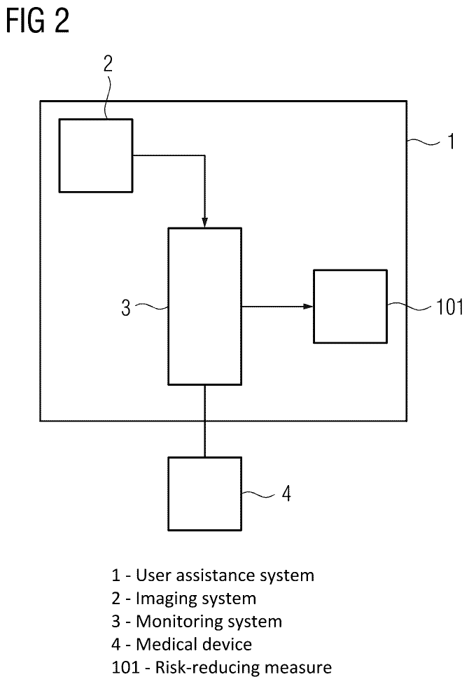

FIG. 2 depicts a block diagram of a further exemplary embodiment of the user assistance system 1 for image support during navigation of the medical device 4. The user assistance system 1 includes the imaging system 2 that is configured, for image support during navigation of the medical device 4, to generate and display the graphical representation of the image data that at least partially maps the medical device 4. The user assistance system 1 also includes the monitoring system 3 that is configured to monitor whether the medical device 4 is moving. The monitoring system 3 is configured to initiate a first risk-reducing measure 101 if the image support by the imaging system 2 is deactivated and, according to the monitoring by the monitoring system 3, the medical device 4 is moving at the same time.

The imaging system 2 may be configured to send status information to the monitoring system 3 in order to ascertain an operating status. This operating status may have at least two states, for example, active and inactive. The monitoring system 3 may be embodied as a data processing unit and thus in particular be configured to detect, store and interlink data. In particular, information about the movement of the medical device 4 may likewise be detected and processed by the monitoring system 3.

The monitoring system 3 may trigger the first measure 101 if two conditions are met. A first condition is that the imaging system 2 is not active, e.g., is deactivated. A second condition is that the medical device 4 is moving. The first measure 101 may warn a user of the user assistance system 1 so that the user, for example, stops the movement of the medical device 4 or activates the imaging system 2. As an escalation act, the first measure 101 may also include an automatic shutdown of the movement. This may take place by deactivating actuators or, for example, also by switching off a supply voltage. It is likewise possible for the monitoring system 3 to activate the imaging system 2, e.g., to enable a graphical representation of the image data.

The first measure 101 is risk-reducing, e.g., it reduces the risk of uncontrolled movement of the medical device 4. The medical device 4 may be located in a body of a human or animal. In this case, uncontrolled movement may lead to injury of the human or animal. On the other hand, controlled movement is a movement of the medical device 4, that takes place with the imaging system activated, e.g., with image support.

For example, during vascular surgery, the medical device 4 may be located in the object 10, in particular in the patient's body, and the image support for navigation of the medical device 4 may be activated. The medical device 4 may include a catheter or a guidewire located in the organ 12 of the patient. The imaging system 2 for image support may be an angiography system. Herein, the graphical representation of the image data is, for example, generated continuously and made available to the user in order to provide assistance with the movement of the medical device 4. On the graphical representation of the image data, the user may recognize the medical device 4 against a vascular background and perform the movement in a targeted manner.

For example, an irregularity may cause the image support to be deactivated so that the user cannot identify progress, or may no longer identify progress, during the movement. If the user continues the movement when the image support is deactivated, the first measure 101 is triggered, for example, in the form of an acoustic signal 150. Likewise, alternatively or additionally, for example, a visual signal 140 may be triggered in the form of a warning message on the display unit 6 which alerts the user to the risk of uncontrolled movement, for example, in text or symbol form.

Likewise, the movement may cause the medical device 4 to move out of an image region of the graphical representation of the image data so that it is no longer visible. If there is movement, the second measure 102 may be triggered in the form of the acoustic signal 150 and/or the visual signal 140. Likewise, the second measure 102 may be triggered if the medical device 4 moves and the image content of the graphical representation of the image data does not meet a predetermined criterion, for example, a quality criterion, such as a signal-to-noise ratio or freedom from artifacts.

The third measure 103 may be initiated by the monitoring system 3 if the user is located in a position where the gaze detection system 5 is unable to detect the gaze of the user, yet the medical device 4 is still moving. In addition, the fourth measure 104 may be initiated by the monitoring system 3 if, although the gaze of the user may be detected by the gaze detection system 5, the gaze is not directed at the display unit 6 and the medical device 4 is also moving.

The monitoring system 3 may receive and evaluate a sensor signal 120 from at least one sensor 9 for monitoring the movement. The at least one sensor 9 may be located directly on or in the medical device 4 and move with it so that the sensor signal 120 may correspond to an actual movement. Likewise, part of the medical device 4 may be located outside the object 10 and the movement may be detected by the monitoring system 3 with a motion detector.

In the described scenarios, the movement of the medical device 4 may take place both manually by the user or automatically actuated by a robotic system. In particular, in the case of the robotic system, one of the measures 101, 102, 103, or 104 may also be the deactivation of at least one actuator 7 and thus a forced stop, e.g., a mandatory termination of the movement of the medical device 4. This may be executed as an escalation measure in order to reduce the risk of injury.

With the robotic system, the monitoring system 3 may monitor the movement of the medical device 4 via a control signal 110. The control signal 110 may be generated by a control device 8 that actuates the at least one actuator 7. In the event that the medical device 4 may only be moved by the at least one actuator 7, the control signal 110 may be a complete indicator of the movement.

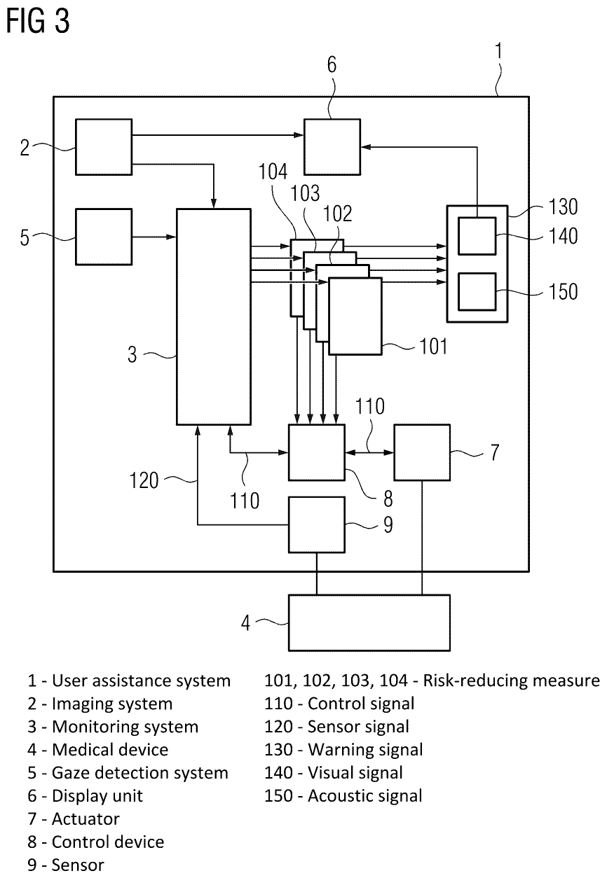

FIG. 3 depicts a block diagram of a further embodiment of the user assistance system 1. The user assistance system 1 is, for example, based on the user assistance system 1 in FIG. 1 and/or the user assistance system 1 in FIG. 2.

The user assistance system 1 may include a gaze detection system 5 that is connected to the monitoring system 3 and may be configured to transmit information about the direction of the gaze of the user to the monitoring system 3.

Based on further conditions, the monitoring system 3 may initiate a second measure 102, a third measure 103 and/or a fourth measure 104, in particular if a corresponding first condition and a corresponding second condition are met simultaneously. The respective second condition may be the same for each of the measures and correspond to the movement of the medical device 4.

A first condition for the second measure 102 may be failure to meet an image quality previously determined by establishing a predetermined criterion. A first condition for the third measure 103 may be the presence of a detection of a gaze direction by the gaze detection system 5. If the gaze detection system 5 does not detect a direction of the gaze of the user and movement of the medical device 4 is detected at the same time, the third measure 103 is initiated. A first condition for the fourth measure 104 may be a comparison of the direction of the gaze of the user with the position of the display unit 6. If the comparison reveals that the gaze of the user is not directed at the display unit 6 and movement of the medical device 4 is detected at the same time, the fourth measure 104 may be initiated.

The initiation of the first, second, third, or fourth measure 101, 102, 103, 104 may be independent of the initiation of other measures 101, 102, 103, 104 in each case and each measure 101, 102, 103, 104 may be initiated individually or in any combination with another measure 101, 102, 103, 104.

The first, second, third, and fourth measures may in each case include issuing at least one warning signal 130 intended to alert the user to the risk of uncontrolled movement of the medical device 4. This may be a visual signal 140 that may be issued by an output device, for example, via the display unit 6. In particular, the visual signal 140 may be depicted at a position of the display unit 6 that was ascertained by the gaze detection system 5. Likewise, the at least one warning signal 130 may include an acoustic signal 150.

In one advantageous embodiment, the individual measures 101, 102, 103, 104 may differ in such a way that the user may easily and quickly deduce the cause of the respective measure 101, 102, 103, 104. In particular, a sequence of different manifestations of the acoustic signal 150 and the visual signal 140 may be determined.

The user assistance system 1 may include at least one sensor 9 that is connected to the medical device 4. In particular, the at least one sensor 9 may also be mechanically connected to the medical device 4 and thus, for example, detect movement. The at least one sensor 9 is connected to the monitoring system 3 and may transmit the sensor signal 120 to the monitoring system 3, which indicates whether or not the medical device 4 is moving, so that, depending on the sensor signal 120 of the at least one sensor 9, the corresponding measure 101, 102, 103, 104 may be initiated.

In a further embodiment, the user assistance system 1 includes at least one actuator 7 that may be controlled via the control device 8. In particular, the medical device 4 may be moved by the at least one actuator 7. In this embodiment, one may speak of automatic movement or robotics. In particular, the movement of the medical device 4 is only possible or provided with the aid of the at least one actuator 7. The control device 8 actuates the at least one actuator 7 with the aid of the control signal 110. The control device 8 and/or the actuator 7 may also provide the control signal 110 to the monitoring system 3 so that the monitoring system 3 may infer from the presence of the control signal 110 that the medical device 4 is about to move.

The first, second, third, and fourth measures 101, 102, 103, 104 may include suppressing the control signal 110 so that, for example, movement of the medical device 4 is not possible as long as the corresponding measure 101, 102, 103, 104 is initiated. In particular, this may also represent an escalation of the at least one warning signal 130 already issued previously.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A user assistance system for image support during navigation of a medical device, the user assistance system comprising:
an imaging system configured, for image support during the navigation of the medical device, to: (1) generate image data that at least partially maps the medical device; and (2) display a graphical representation of the image data; and
a monitoring system configured to: (1) monitor whether the medical device is moving; and (2) initiate a first risk-reducing measure when the image support by the imaging system is deactivated and, according to the monitoring by the monitoring system, the medical device is moving at a same time.

2. The user assistance system of claim 1, wherein the imaging system is a medical imaging system for mapping the medical device in an organ and/or tissue.

3. The user assistance system of claim 1, wherein the monitoring system is configured to:
check an image content of the image data and/or the graphical representation of the image data for a predetermined criterion; and
initiate a second risk-reducing measure when the image content does not meet the predetermined criterion and, according to the monitoring by the monitoring system, the medical device is moving at the same time.

4. The user assistance system of claim 1, further comprising:
a gaze detection system configured to detect a gaze direction of a user,
wherein the monitoring system is configured to initiate a third risk-reducing measure and/or a fourth risk-reducing measure depending on a result of the detecting of the gaze direction.

5. The user assistance system of claim 4, wherein the monitoring system is configured to initiate the third risk-reducing measure when the gaze detection system does not detect the gaze direction of the user and, according to the monitoring by the monitoring system, the medical device is moving at the same time.

6. The user assistance system of claim 5, further comprising:
a display unit configured to display the graphical representation of the image data,
wherein the monitoring system is configured to initiate the fourth risk-reducing measure when the gaze detection system determines that the gaze direction of the user is not directed at the display unit and, according to the monitoring by the monitoring system, the medical device is moving at the same time.

7. The user assistance system of claim 4, further comprising:
a display unit configured to display the graphical representation of the image data,
wherein the monitoring system is configured to initiate the fourth risk-reducing measure when the gaze detection system determines that the gaze direction of the user is not directed at the display unit and, according to the monitoring by the monitoring system, the medical device is moving at the same time.

8. The user assistance system of claim 1, further comprising:
at least one actuator configured to move the medical device.

9. The user assistance system of claim 8, further comprising:
a control device configured to: generate at least one control signal for actuating the at least one actuator; and provide the at least one control signal to the at least one actuator and to the monitoring system,
wherein the monitoring system is configured to monitor, depending on the control signal, whether the medical device (4) is moving.

10. The user assistance system of claim 8, wherein the first risk-reducing measure comprises deactivating the at least one actuator.

11. The user assistance system of claim 1, further comprising:
at least one sensor configured to issue a sensor signal depending on a movement of the medical device,
wherein the monitoring system is configured to monitor whether the medical device is moving depending on the sensor signal.

12. The user assistance system of claim 1, wherein the first risk-reducing measure comprises issuing at least one warning signal.

13. The user assistance system of claim 12, wherein the at least one warning signal comprises a visual signal, and
wherein the user assistance system is configured to display the visual signal.

14. The user assistance system of claim 13, further comprising:
a gaze detection system configured to detect a gaze direction of a user; and
a display unit configured to display: (1) the graphical representation of the image data; and (2) the visual signal at a display position that lies in the detected gaze direction of the user,
wherein the monitoring system is configured to initiate a third risk-reducing measure when the gaze detection system does not detect the gaze direction of the user and, according to the monitoring by the monitoring system, the medical device is moving at the same time, or
wherein the monitoring system is configured to initiate a fourth risk-reducing measure when the gaze detection system determines that the gaze direction of the user is not directed at the display unit and, according to the monitoring by the monitoring system, the medical device is moving at the same time.

15. The user assistance system of claim 12, wherein the at least one warning signal comprises an acoustic signal.

16. A method for image support during navigation of a medical device, the method comprising:
generating, by an imaging system, a graphical representation of image data that at least partially maps the medical device;
displaying, by the imaging system, the graphical representation of the image data;
monitoring, by a monitoring system whether the medical device is moving; and
initiating, by the monitoring system, a first risk-reducing measure when the image support by the imaging system is deactivated and, according to the monitoring by the monitoring system, the medical device is moving at a same time.
